(19) United States
(12) Patent Application Publication
(10) Pub. No.: US 2020/0297944 A1
(43) Pub. Date: Sep. 24, 2020

(54) PERSONAL MEASURED DOSING DEVICE AND METHOD

(71) Applicants: Robert LaChance, Pasadena, CA (US); Ronald Seley, Pasadena, CA (US)

(72) Inventors: Robert LaChance, Pasadena, CA (US); Ronald Seley, Pasadena, CA (US)

(21) Appl. No.: 16/360,849

(22) Filed: Mar. 21, 2019

(51) Int. Cl. A61M 11/00; A61M 15/00; A61M 11/04; A24F 47/00

(52) U.S. Cl. CPC A61M 11/005; A61M 15/0065; A61M 15/0001; A24F 47/008; A61M 11/042

(57) ABSTRACT

A hand-held personalized measured dosing inhalation device having one or more cartridges containing vaporizable-liquid, one or more corresponding atomizers configured to vaporize the liquid so as to be inhaled by a user, a micro controller configured to monitor and regulate the vaporization process at one or more of the cartridges, and one or more external biometric sensors configured to be in communication with the controller, wherein the controller regulates vaporization of the liquid only upon authorization conveyed by the biometric sensors and when predetermined conditions are satisfied, the authorization condition being partially determined by input signaling received from internal sensors coupled to the cartridges and/or outlet.

### FIELD OF THE INVENTION

The present disclosure relates to hand-held personalized measured dosing inhalation devices.

### DESCRIPTION OF RELATED ART

In the context of popular consumer usage, vaporizing is the conversion of a liquid into a vapor to be inhaled by a user. When compared to burning of physical fuel, vaporizing is an alternative method of creating an inhalable compound that avoids the unpleasing inhalation of many irritating and unhealthy by-products. Vaporizing devices most often include components and/or structures for causing fluids to vaporize, including, but not limited to, ultrasonic devices that use ultrasound waves to change fluid into vapor, and heating devices that use a rapid change in temperature to fluids in close proximity to a heating element or surface into vapor.

Because of their pleasant usage characteristics and simplicity, the use of vaporizing inhalation devices are now commonplace. Most often, vaporizing inhalation devices are recognized as electrically-powered heating-element-driven vaporizers (or “vaping devices”), which simulate the act of smoking without directly burning solid materials like processed and treated tobacco leaves. In the case of replacing or supplanting tobacco and/or nicotine delivery, these vaporizers are widely recognized as electronic cigarettes, and are considered a boon for those attempting to quit smoking traditional cigarettes. Although innovative when first introduced, the limitations of such devices are now apparent, such as their reliance on a singular vaporization chamber and fluid reservoir that limited the user to only a single type of vapor for that single type of fluid reservoir.

Vaping devices are also frequently used as an alternative tool for delivering medicines to ailing patients. The benefits enjoyed by recreational users of vaping devices' are equally applicable to those seeking medical help, where vaporized drugs may be ingested more pleasantly and effectively than orally, through combustion-based inhalation, or even intrusive and painful injections or IV drips. Device designs incorporating multiple vaporization reservoirs and/or chambers would be a benefit in the medical field as well, as flavorings or relaxants could be vaporized in careful measure alongside any medication to mask of eliminate unpleasant tastes or sensations during ingestion.

Although widespread, the use of vaping devices is also still considered a recent phenomenon. There is not very much evidence demonstrating adverse health effects from inhaling vapor as a recreational activity or even medicinal treatment, in comparison to other forms of substance ingestion. Due to the nature of some recreational inhalants and prescribed medicinal drugs, there is a concern that unrestricted access to such materials could prove dangerous without professional guidance, oversight, and/or restrictions. Conventional vaping devices do not appear to adequately address concerns with controlled access to such substances.

Overall, the vaping devices known today are limited in use, limited in security, limited in dosage control, and limited in safety. It is desirable to have a hand-held personalized measured dosing inhalation device that not only eases the ingestion of controlled, prescription, or other medicinal substances, but does so in a regulated manner, while allowing for a controlled combination of a variety of constituent fluids to produce a uniquely-tailored vapor for each user.

### BRIEF SUMMARY OF THE INVENTION

According to various embodiments of the present invention, a hand-held personalized measured dosing inhalation device is provided with one or more biometric sensors that allow the personal vaporizer to operate, and thus vaporize a liquid material, for only a particular user.

According to other embodiments, a hand-held personalized measured dosing inhalation device is provided with multiple replaceable cartridges, each themselves having multiple internal chambers in which different liquid materials may be respectively stored and subsequently vaporized in a condition of authorized usage.

According to other embodiments, a hand-held personalized measured dosing inhalation device is provided wherein a third party, including but not limited to a licensed medical professional, is able to remotely control the various vaporization characteristics of a single and/or multi-chambered device to prevent unauthorized vaporization, harm to a user, and/or ensure adherence to a prescribed dosing regimen.

In some embodiments, a hand-held personalized measured dosing inhalation device is provided wherein it includes a rechargeable power source, integrated printed circuit board (PCB), and/or wireless communication capability that allows for direct interaction by a user and/or third party with the control schema of the device via a software application on a portable computing device, an application on a terminal computing device, a browser-based interface, or another associated graphic user interface.

In still other embodiments, the hand-held personalized measured dosing inhalation device can appear as a traditional medical-use inhaler with protruding front inhalation mouthpiece. In other embodiments, a hand-held personalized measured dosing inhalation device can appear as an elongate or cylindrical device, akin to a traditional cigarette or cigar.

According to other embodiments, the hand-held personalized measured dosing inhalation device, either by itself through embedded and preprogrammed software checks and sensors or in concert with a remotely-controlled and accessed software application, can control the application of vaporizing energy (whether in the form of ultrasonic waves or heat applied via a physical coil or element) supplied by an internal power supply, so as to optimize predetermined dosing and/or vaporization characteristics.

According to further embodiments, cartridges for use with the hand-held personalized measured dosing inhalation device can be replaceable, refillable, and/or available in various volumetric capacities. Each such cartridge can also be tagged with a unique identifier, which the device can recognize as authorized, unauthorized, or as requiring certain dosing and vaporization constraints or characteristics be applied when vaporizing materials contained therein.

In another embodiment, the hand-held personalized measured dosing inhalation device may include sensors, wireless transceivers, and storage media for detecting, recording, and transmitting vaporization usage cycle characteristics in comparison to and/or combination with the identifying information gleaned from the installed cartridges. This information can then be either displayed an/or used by software embedded within the device itself, or by a user or third partly remotely via a wireless graphic user interface, to control the vaporization usage and/or dosing provided by the device, including but not limited to, entering a conditional period of locking or restriction given a particular usage cycle, and alerting a user or third party of a impending depletion of fluid in an installed cartridge given its initial capacity in comparison to historical usage cycles and vaporization rates.

Additional aspects, features, and advantages of the present invention will be apparent in part from the description, drawings, claims that follow, and in part will become apparent to those skilled in the art upon examination of the following, or may be learned from the practice of the invention.

### BRIEF DESCRIPTION OF THE DRAWINGS

The above and other aspects, features and advantages of the present invention will be more apparent from the following more particular description thereof, presented in conjunction with the following drawings wherein:

FIG. 1 illustrates an exploded view of a hand-held personalized measured dosing inhalation device according to an example embodiment bearing a single vaporization chamber and fluid reservoir;

FIG. 2 illustrates a front plan exploded view of a hand-held personalized measured dosing inhalation device according to an example embodiment bearing a single vaporization chamber and fluid reservoir;

FIG. 3 illustrates a bottom plan view of a hand-held personalized measured dosing inhalation device according to an example embodiment bearing a single vaporization chamber and fluid reservoir;

FIG. 4 illustrates a cross-section view of a hand-held personalized measured dosing inhalation device according to an example embodiment bearing a single vaporization chamber and fluid reservoir;

FIG. 5 illustrates a perspective view of a hand-held personalized measured dosing inhalation device according to an example embodiment bearing a single vaporization chamber and fluid reservoir;

FIG. 6 illustrates an exploded view of a hand-held personalized measured dosing inhalation device according to an example embodiment bearing two vaporization chambers and fluid reservoirs;

FIG. 7 illustrates a front plan exploded view of a hand-held personalized measured dosing inhalation device according to an example embodiment bearing two vaporization chambers and fluid reservoirs;

FIG. 8 illustrates a bottom plan view of a hand-held personalized measured dosing inhalation device according to an example embodiment bearing two vaporization chambers and fluid reservoirs;

FIG. 9 illustrates a cross-section view of a hand-held personalized measured dosing inhalation device according to an example embodiment bearing two vaporization chambers and fluid reservoirs; and

FIG. 10 illustrates a perspective view of a hand-held personalized measured dosing inhalation device according to an example embodiment bearing two vaporization chambers and fluid reservoirs.

### DETAILED DESCRIPTION

The present invention comprising a variety of a hand-held personalized measured dosing inhalation device embodiments will now be described. In the following exemplary description numerous specific details are set forth in order to provide a more thorough understanding of embodiments of the invention. It will be apparent, however, to an artisan of ordinary skill that the present invention may be practiced without incorporating all aspects of the specific details described herein. Furthermore, although steps or processes are set forth in an exemplary order to provide an understanding of one or more systems and methods, the exemplary order is not meant to be limiting. One of ordinary skill in the art would recognize that the steps or processes may be performed in a different order, and that one or more steps or processes may be performed simultaneously or in multiple process flows without departing from the spirit or the scope of the invention. In other instances, specific features, quantities, or measurements well known to those of ordinary skill in the art have not been described in detail so as not to obscure the invention. It should be noted that although examples of the invention are set forth herein, the claims, and the full scope of any equivalents, are what define the metes and bounds of the invention.

For a better understanding of the disclosed embodiment, its operating advantages, and the specified object attained by its uses, reference should be made to the accompanying drawings and descriptive matter in which there are illustrated exemplary disclosed embodiments. The disclosed embodiments are not intended to be limited to the specific forms set forth herein. It is understood that various omissions and substitutions of equivalents are contemplated as circumstances may suggest or render expedient, but these are intended to cover the application or implementation.

The term “first”, “second” and the like, herein do not denote any order, quantity or importance, but rather are used to distinguish one element from another, and the terms “a” and “an” herein do not denote a limitation of quantity, but rather denote the presence of at least one of the referenced item.

Spatially relative terms, such as “beneath,” “below,” “lower,” “under,” “above,” “upper,” and the like, may be used herein for ease of explanation to describe one element or feature's relationship to another element(s) or feature(s) as illustrated in the figures, it will be understood that the spatially relative terms are intended to encompass different orientations of the device in use or in operation, in addition to the orientation depicted in the figures. For example, if the device in the figures is turned over, elements described as “below” or “beneath” or “under” other elements or features would then be oriented “above” the other elements or features. Thus, the example terms “below” and “under” can encompass both an orientation of above and below. The device may be otherwise oriented (e.g., rotated 90 degrees or at other orientations) and the spatially relative descriptors used herein should be interpreted accordingly.

It will be understood that when an element or layer is referred to as being “on,” “connected to,” or “coupled to” another element or layer, it can be directly on, connected to, or coupled to the other element or layer, or one or more intervening elements or layers may be present. In addition, it will also be understood that when an element or layer is referred to as being “between” two elements or layers, it can be the only element or layer between the two elements or layers, or one or more intervening elements or layers may also be present.

As used herein, the term “substantially,” “about,” and similar terms are used as terms of approximation and not as terms of degree, and are intended to account for the inherent deviations in measured or calculated values that would be recognized by those of ordinary skill in the art. Further, the use of “may” when describing embodiments of the present invention refers to “one or more embodiments of the present invention.” As used herein, the terms “use,” “using,” and “used” may be considered synonymous with the terms “utilize,” “utilizing,” and “utilized,” respectively. Also, the term “exemplary” is intended to refer to an example or illustration.

Unless otherwise defined, all terms (including technical and scientific terms) used herein have the same meaning as commonly understood by one of ordinary skill in the art to which the present invention belongs. It will be further understood that terms, such as those defined in commonly used dictionaries, should be interpreted as having a meaning that is consistent with their meaning in the context of the relevant art and/or the present specification, and should not be interpreted in an idealized or overly formal sense, unless expressly so defined herein.

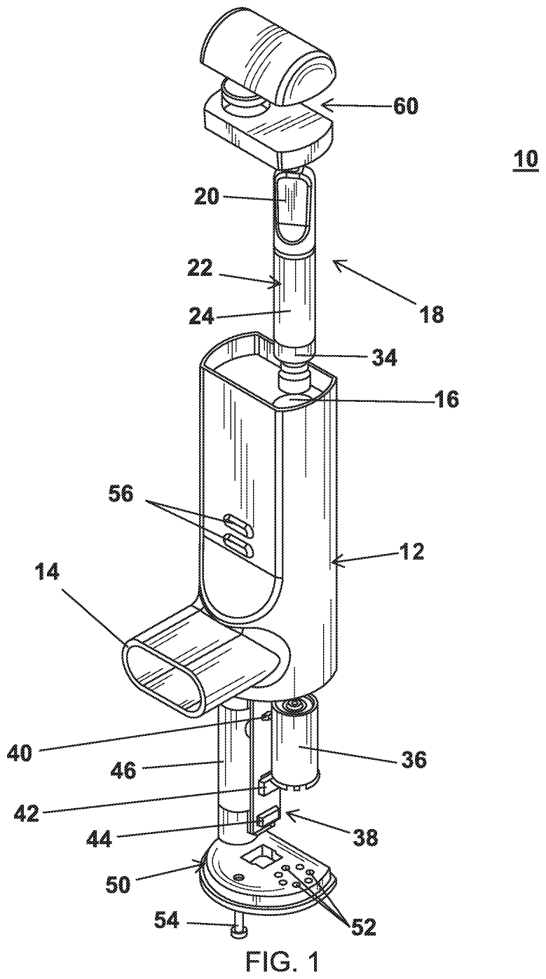

One or more embodiments of the present invention will now be described with references to FIGS. 1-10.

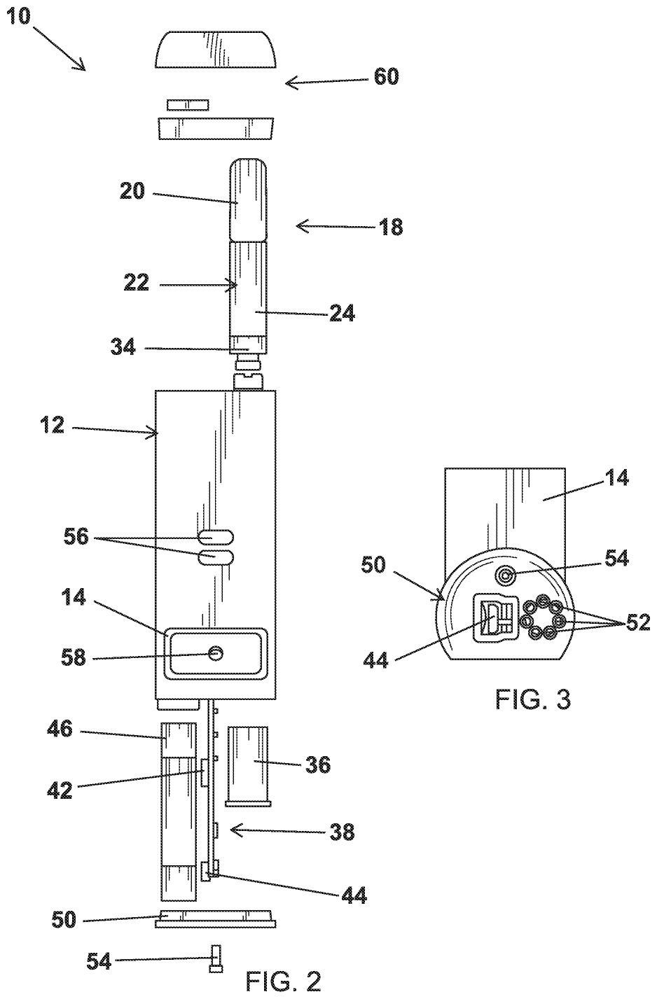

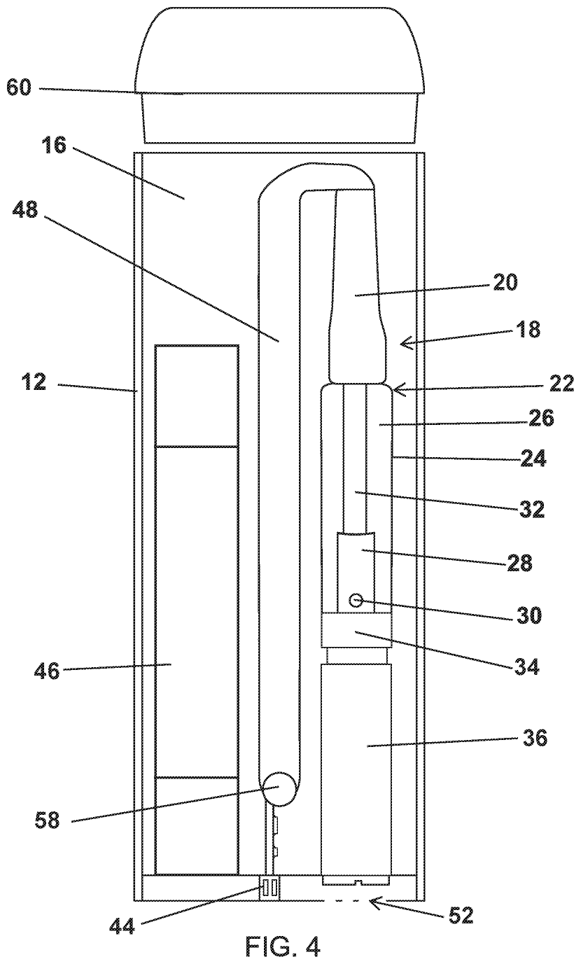

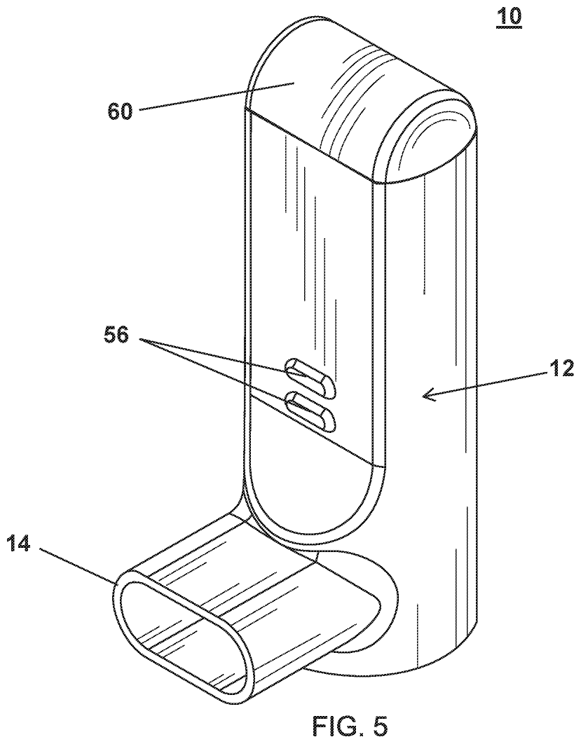

FIGS. 1 and 2 illustrate exploded views of one embodiment of a hand-held personalized measured dosing inhalation device (“Inhalation Device”) 10. The Inhalation Device 10 includes an outer body 12 having a mouthpiece 14, internal cavity 16 sufficiently-sized to house a vaporization cartridge assembly 18, an atomizer duty controller 36, a micro controller 38, a power source 46, and a contoured vapor venting chamber 48 (shown in FIG. 4), a bottom plate 50 having breather holes 52, a compression-fit coupler 54 for securing the bottom plate 50 to the outer body 12, biometric sensors 56 visible on the outer body 12 above the mouthpiece 14, and a removable convex top cap 60. The outer body 12 is shown in FIG. 1 as having a substantially rectangular shape with bulging outer minor surfaces and substantially flat outer major surfaces, where the substantially rectangular or ovoid mouthpiece 14 protrudes perpendicularly from a lower portion—relative to its closer proximity to the bottom plate 50—of a major flat surface of the outer body 12. FIG. 5 illustrates a non-exploded perspective view of the Inhalation Device 10, again clearly exhibiting the perpendicular protrusion of the mouthpiece 14 from the outer body 12. In another embodiment, the outer body 12 and mouthpiece 14 may be axially-aligned along the length of the outer body 12, such that the overall visual impression of the Inhalation Device 10 is that of a rectangular cuboid, cylinder, or similar three-dimensional elongate structure.

The vaporization cartridge assembly 18 is comprised of a contoured venting port 20, a fluid reservoir assembly 22, and a conductive tubular interface 34 electrically connected to a heating vaporization element (not shown) housed within a vaporizing element 28 (shown in FIG. 4). The fluid reservoir assembly 22 comprises an outer substantially-cylindrical shell 24 having an internal cavity 26 (shown in FIG. 4), the vaporizing element 28 (shown in FIG. 4) with a fluid interface port 30 (shown in FIG. 4), and an internal vaporization transfer tube 32 connected to the contoured venting port 20. In some embodiments, the vaporization cartridge assembly 18 is a replaceable non-refillable consumable component with a fixed amount of fluid stored in its internal cavity 26 (shown in FIG. 4). In alternative embodiments, the vaporization cartridge assembly 18 is a removable refillable component with a user-controlled—and user-reported to the controller chip 42 on the micro controller 38—fluid amount stored in the internal cavity 26 (shown in FIG. 4).

To remove, replace, and/or refill the vaporization cartridge assembly 18, a user or third party would remove the top cap 60 from the outer body 12, and pull the vaporization cartridge assembly 18 out of its seat with the atomizer duty controller 36 using mechanical force. In an alternative embodiment, the vaporization cartridge assembly 18 would only be removable via a twisting or unscrewing movement, as it would be seated in the atomizer duty controller 36 using a threaded, rather than press-fit, connector. In another embodiment, the vaporization cartridge assembly 18 would be locked into its installed position upon insertion to its seat on the atomizer duty controller 36, via clamping mechanical actuators (not shown) controlled by the micro controller 38. The micro controller 38 may sense the type of cartridge 18 installed, and thus detect° and record information related to its fluidic contents, and would then be able to determine a locking regime to prevent unauthorized removal of a particular vaporization cartridge assembly 18 as determined by vaporization dosing and usage parameters set by a user, third-party, or both. Such vaporization dosing and usage parameters include, but are not limited to, the calculated approximate amount of remaining fluid in the cartridge 18 based on an initial known level as conveyed by sensed cartridge 18 identifying information upon cartridge 18 installation, the fluid within the cartridge 18 as conveyed by sensed cartridge 18 identifying information upon cartridge 18 installation, and prescribed overall Inhalation Device 10 usage amounts and deadlines.

The micro controller 38 exhibited in the embodiment shown in FIG. 1 includes at least a wireless transceiver 40 capable of transmitting and receiving information via one or more wireless communication protocols such as WiFi or Bluetooth, a controller chip 42 for storing operative controls, parameters, commands, and settings, managing and recording various internal sensor (not shown in FIG. 1) inputs, and managing the conduction of electricity from the power source 46 to the atomizer duty controller 36 such that vaporization characteristics are carefully managed, and an externally-accessible connection port 44 to allow for communicative connection to a remote computer (not shown) and/or to recharge the internal power source 46. The micro controller 38 is also communicatively-coupled to the biometric sensors 56 on the outer body 12, such that a user may provide a biometric identifier to the sensors 56 and gain access to a predetermined vaporization dosing amount from an installed vaporization cartridge assembly 18 during use of the Inhalation Device 10. In an alternative embodiment, the power source 46 is not rechargeable, but instead a replaceable consumable power source, such as AA, AAA, C, D, and/or 9V batteries that are widely available for purchase.

In one embodiment, the biometric sensors 56 are instead a single sensor (not shown). In another embodiment, the biometric sensors 56 sense identifying fingerprinting information. In yet another embodiment, the biometric sensors 56 sense iris or facial identifying information. In some embodiments, the biometric sensors 56 incorporate lighting elements (not shown), such as LEDs (not shown) embedded beneath a clear outer detection cover (not shown) that can convey information through the application of a variety of colors, intermittent illumination regimens, or both, about the status of the Inhalation Device 10, including but not limited to its locked or unlocked status, approval of the provided identifying information via the biometric sensors 56, power supply 46 capacity and status, and estimated fluid amount remaining in an installed and identified vaporization cartridge assembly 18.

When fully assembled, the Inhalation Device 10 allows for the pressurized communication of atmospheric air through the breather holes 52 such that air and/or vapor exits the contoured vapor venting chamber 48 at an outlet 58 (shown in FIGS. 2 & 4) within the mouthpiece 14.

FIG. 3 illustrates a bottom plan view of an embodiment of the present invention, which highlights one possible place and configuration of the breather holes 52 in the bottom plate 50, along with the externally-accessible connection port 44. In another embodiment, the breather holes 52 may instead comprise a single large vented opening with a mesh protector to filter any unwanted detritus from entering the Inhalation Device 10. The connection port 44 may be a USB port, MicroUSB port, MiniUSB port, firewire port, or any other standards-approved connection to the micro controller 38 that allows for the communication of data and the transmission of power to charge a rechargeable power supply 46 (shown in FIGS. 1, 2, & 4).

FIG. 4 illustrates a cross-sectional view of an embodiment of the present invention. Upon an application of negative pressure at the mouthpiece 14 (shown in FIG. 1) by a user, a negative pressure differential is created at the outlet 58 such that the fluidic communication of the atmospheric air throughout the Inhalation Device 10 starting at the breather holes 52, allows air to flow from the surrounding atmosphere through the holes 52, into the atomizer duty controller 36, up through the conductive tubular interface 34, into the vaporizing element 28, and then through the internal vaporization transfer tube 32, through the contoured venting port 20, into the contoured vapor venting chamber 48, and finally down to the outlet 58 where it exits the Inhalation Device 10 via the mouthpiece 14. Valving (not shown) may ensure that fluid stored within the internal cavity 26 of the fluid reservoir assembly 22 does not travel through the vaporizing element 28 up into the internal vaporization transfer tube 32 via the fluid interface port 30 upon the same application of negative pressure at the outlet 58.

Upon the same application of negative pressure at the outlet 58, the micro controller 38, having received confirmation of approved usage through the identifying information conveyed by the biometric sensors 56, and having confirmed that historical vaporization data recorded by the controller chip 42 and preset usage conditions set by either the user, a third party, or both, allows for the application of electrical current from the power source 46 to heat a vaporizing heating element (not shown) within the vaporizing element 28, to excite the fluid (not shown) within the vaporizing element 28 in the presence of flowing air, thus creating vapor. As explained in the air-only context above, that same negative pressure would then pull the vapor out of the vaporizing element 28, through the internal vaporization transfer tube 32, through the contoured venting port 20, into the contoured vapor venting chamber 48, and finally down to the outlet 58 where it exits the Inhalation Device 10 via the mouthpiece 14 (shown in FIG. 1).

In one embodiment, the micro controller 38 may limit the duration of the application of electrical power from the power source 46 to the heating element (not shown) in the vaporizing element 28. In an alternative embodiment, the micro controller 38 controls the transfer of electrical power from the power source 46 to the heating element (not shown) in the vaporizing element 28, based on the preset parameters of the vaporization quality desired by the user, as indicated via a user interface accessible remotely in a software application, internet browser, or other interface removed from the Inhalation Device 10 itself. In a further alternative embodiment, the micro controller 38 controls the transfer of electrical power from the power source 46 to the heating element (not shown) in the vaporizing element 28, based on the preset parameters of the vaporization quality desired by the user. In a further alternative embodiment, the micro controller 38 receives instructions remotely from a user and/or third party via the wireless transceiver 40, which then allows for the measured timing control on the transfer of electrical power from the power source 46 to the heating element (not shown) in the vaporizing element 28. In a further alternative embodiment, the micro controller 38 transmits sensor readings and calculated approximated status readings to a user and/or third party via the wireless transceiver 40, which then allows for the user and/or third party to appraise the vaporization use of the Inhalation Device 10 and determine whether dosing vaporization controls should be changed. In a further alternative embodiment, the micro controller 38 records sensor readings and compares to calculated approximated status readings to automatically regulate vaporization use of the Inhalation Device 10, per preset conditions and parameters defined by a user and/or third-party. In a further alternative embodiment, the micro controller 38 transmits sensor readings and calculated approximated status readings to a user and/or third party via the wireless transceiver 40, which then allows for the user and/or third party to appraise the vaporization use of the Inhalation Device 10 and determine whether dosing vaporization controls should be changed. In some embodiments, the micro controller 38 may also manage the actuation of sealing control valves (not shown) installed in the air-flow path within the Inhalation Device 10, such as within the interface points between the contoured venting port 20 and the contoured vapor venting chamber 48, or the contoured vapor venting chamber 48 and outlet 58 to regulate the flow of air or any other fluid through the device under predetermined criteria, regardless of the type of vaporization cartridge assembly 18 is installed.

In an alternative embodiment, the vaporizing element 28 includes an ultrasonic wave generator (not shown) in place of a heating element (not shown), such that fluid in proximate contact with the ultrasonic wave generator (not shown) converts to vapor upon application of electrical power from the power source 46 to the vaporizing element 28, as managed by the micro controller circuitry 38. This alternative embodiment has the advantage of limiting the risk of overheating or unwanted combustion resulting from generation of excess heat, as an ultrasonic wave generator generates only a minimal amount of heat in its operation.

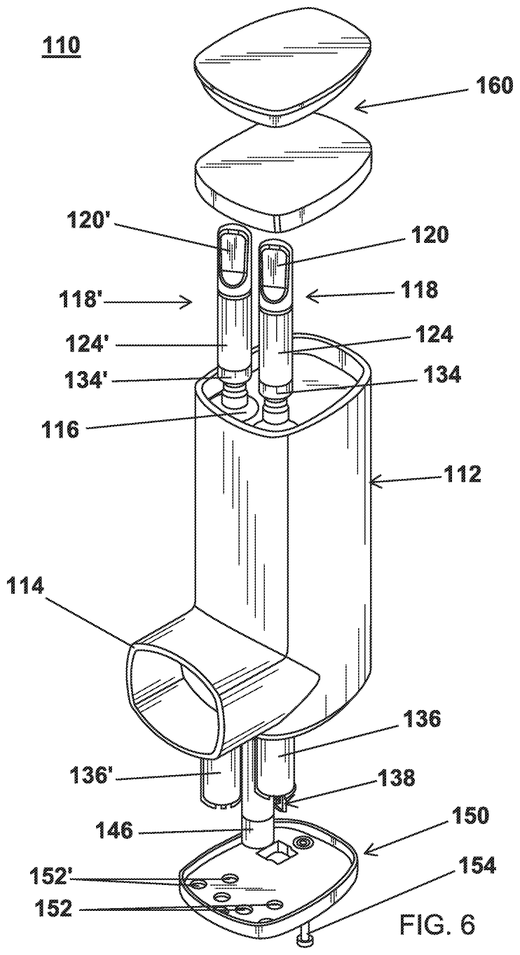

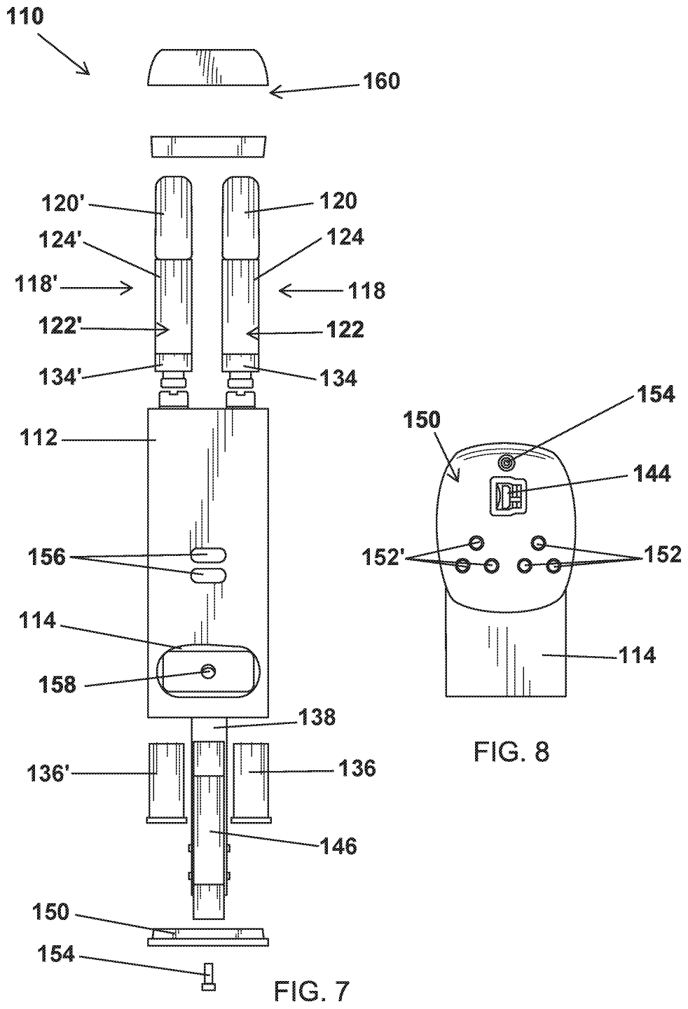

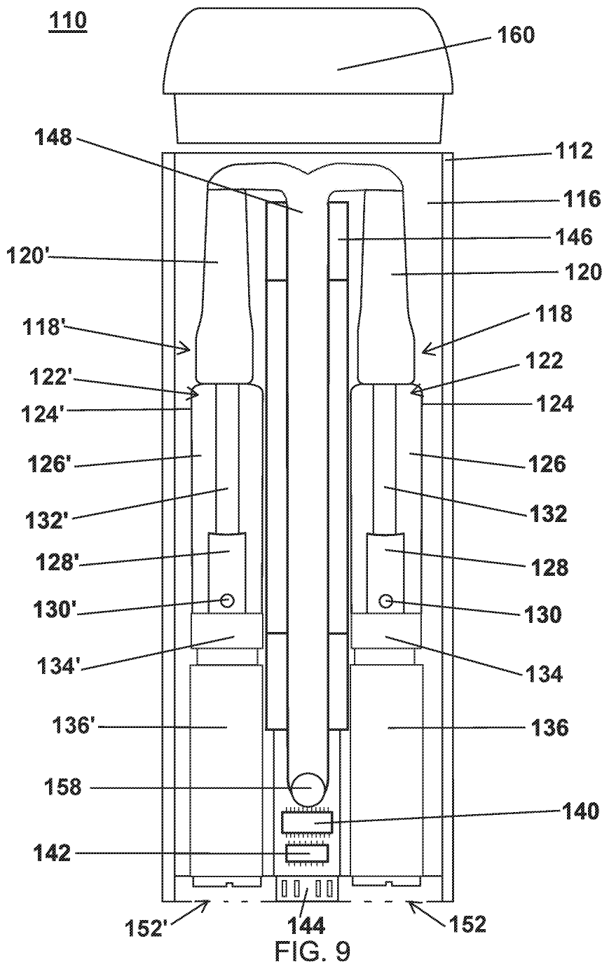

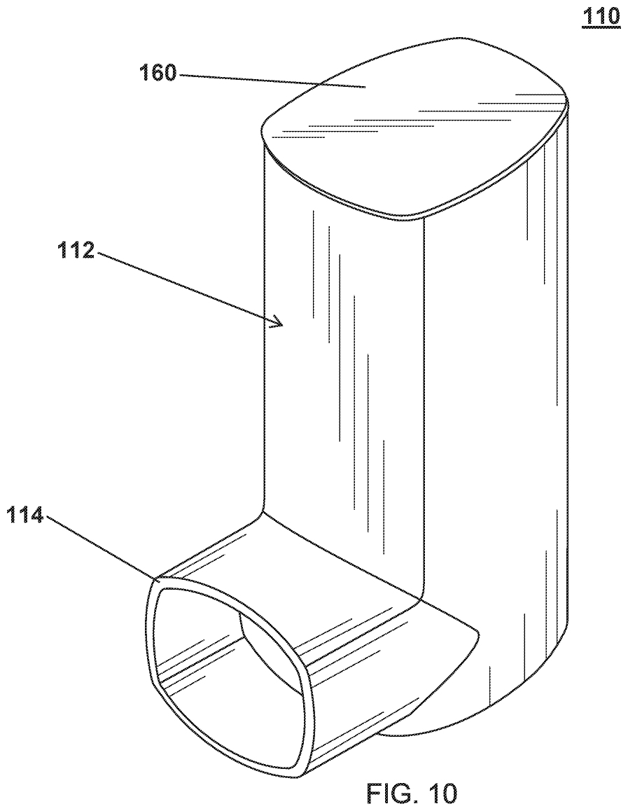

FIGS. 6 & 7 illustrate exploded views of an alternative embodiment of a hand-held personalized measured dosing inhalation device (“Inhalation Device”) 110. The Inhalation Device 110 includes an outer body 112 having a mouthpiece 114, internal cavity 116 sufficiently-sized to house two vaporization cartridge assemblies 118 and 118′, two atomizer duty controllers 136 and 136′, a micro controller 138, a power source 146, and a y-branched vapor venting conduit 148 (shown in FIG. 9), a bottom plate 150 having two sets of breather holes 152 and 152′, a bottom coupler 154 for securing the bottom plate 150 to the outer body 112, biometric sensors 156 (shown in FIG. 7) visible on the outer body 112 above the mouthpiece 114, and a two-piece removable top cap 160. The outer body 112 is shown in FIG. 6 as having a substantially elongated cuboid shape with rounded outer surfaces, where the substantially rectangular or ovoid mouthpiece 114 protrudes perpendicularly from a lower portion—relative to its closer proximity to the bottom plate 150—of the outer body 112. FIG. 10 illustrates a non-exploded perspective view of the Inhalation Device 110, again clearly exhibiting the perpendicular protrusion of the mouthpiece 114 from the outer body 112. In another embodiment, the outer body 112 and mouthpiece 114 may be axially-aligned along the length of the outer body 112, such that the overall visual impression of the Inhalation Device 110 is that of a cuboid, cylinder, or similar three-dimensional elongate structure.

The two vaporization cartridge assemblies 118 and 118′ are each comprised of a contoured venting port 120 and 120′, a fluid reservoir assembly 122 and 122′, and a conductive tubular interface 134 and 134′, each electrically connected to a heating vaporization element (not shown) housed within their respective vaporizing elements 128 and 128′ (shown in FIG. 9). Each fluid reservoir assembly 122 and 122′ comprises outer substantially-cylindrical shells 124 an 124′ having internal cavities 126 and 126′ (shown in FIG. 9), respective vaporizing elements 128 and 128′ (shown in FIG. 9) each with at least one fluid interface port 130 and 130′ (shown in FIG. 9), and internal vaporization transfer tubes 132 and 132′ connected to each respective contoured venting port 120 and 120′. The vaporization cartridge assemblies 118 and 118′ may be replaceable non-refillable consumable component with a fixed amount of fluid, o alternatively may be removable refillable components with a user-controlled fluid amount stored in the internal cavities 126 and 126′ (shown in FIG. 9).

In another embodiment, the vaporizing cartridge assemblies 118 and 118′ are designed to be used in a complementary fashion, and thus include identifying information conveying this complimentary arrangement to the micro circuit controller 138. Upon detection of one cartridge 118 without the other 118′, or vice versa, the micro circuit controller 138 may stop the delivery of any electrical power to the vaporizing element 128 of the singularly-installed cartridge 118 upon negative pressure applied at the outlet 158 (shown in FIGS. 7 and 9). In an alternative embodiment, the cartridges 118 and 118′ may not be identifiable as requiring complementary simultaneous installation, but instead each be separately identifiable via sensors (not shown) detecting embedded identifiable information within the cartridges 118 or 118′ as limiting or changing overall Inhalation Device 110 vaporization characteristics given the presence of the other installed cartridge 118′ or 118, as determined by preset programming by a user, third-party, or both, stored in a controller chip 140 included on the micro controller circuitry 138.

To remove, replace, and/or refill either vaporization cartridge assembly 118 or 118′, a user or third party would remove the two-piece top cap 160 from the outer body 112, and pull the vaporization cartridge assemblies 118 and 118′ out of their seats with each respective atomizer duty controller 136 or 136′ using mechanical force, or alternatively via a twisting or unscrewing movement, as each would be seated in the respective atomizer duty controllers 136 and 136′ using threaded, rather than press-fit, connectors. In another embodiment, each vaporization cartridge assembly 118 and 118′ would be locked into its respective installed position upon insertion via mechanical actuators (not shown) controlled by the micro controller 138. The micro controller 138 may sense the type of cartridges 118 or 118′ installed, and thus detect and record information related to their combined and respective fluidic contents, and would then be able to determine a locking regime to prevent unauthorized removal of a particular vaporization cartridge assembly 118 or 118′ as determined by vaporization dosing and usage parameters set by a user, third-party, or both. Such vaporization dosing and usage parameters include, but are not limited to, the calculated approximate amount of remaining fluid in the cartridges 118 or 118′ based on an initial known level as conveyed by sensed cartridge identifying information upon cartridge 118 or 118′ installation, the composition of the fluid within each cartridge 118 and 118′ as conveyed by sensed cartridge identifying information upon cartridge 118 and 118′ installation, and prescribed overall Inhalation Device 110 usage amounts and deadlines. In another embodiment, the Inhalation Device 110 includes more than two vaporization cartridge assemblies 118 and 118′, where the number of cartridges 118 is only limited by the size and shape of the internal cavity 116 as defined by the shape of the outer body 112, the increases in power consumption from having more vaporization elements 128, and the constraints on the throughput and processing power of the controller chip 140 on the micro circuit controller 138 wherein the micro controller circuit 138 would be taxed by increased input from an increased number of internal sensors (not shown), and the calculation, regulation, and securitized lock-out for complex and potentially-harmful vaporization configurations that may result from having more cartridges 118 installed.

As with the micro controller 38 exhibited in the embodiment shown in FIG. 1 the micro controller circuitry 138 here includes at least a wireless transceiver (not shown) capable of transmitting and receiving information via one or more wireless communication protocols such as WiFi or Bluetooth, a controller chip 142 for storing operative controls, parameters, commands, and settings, managing and recording various internal sensor (not shown in FIGS. 6-10) inputs, and managing the conduction of electricity from the power source 146 to the cartridges 118 and 118′ via the atomizer duty controllers 136 and 136′ such that vaporization characteristics are carefully managed, and an externally-accessible connection port 144 to allow for communicative connection to a remote computer (not shown) and/or to recharge the internal power source 146. The micro controller 138 is also communicatively-coupled to the biometric sensors 156 on the outer body 112, such that a user may provide a biometric identifier to the sensors 156 and gain access to a predetermined vaporization dosing amount from an installed vaporization cartridge assembly 118 or 118′ during use of the Inhalation Device 110. In an alternative embodiment, the power source 146 is not rechargeable, but instead a replaceable consumable power source, such as AA, AAA, C, D, and/or 9V batteries that are widely available for purchase. In an alternative embodiment, the power source 146 may be comprised of an electrically-coupled collection of more than one individual source and/or cell. A multi-cell power source (not shown) may be advantageous and desirous given the increased power requirements of driving the duty cycles of more than one vaporizing element 128 in a multi-cartridge-design embodiment of the present invention.

In one embodiment, the biometric sensors 156 are instead a single sensor (not shown). In another embodiment, the biometric sensors 156 sense identifying fingerprinting information. In yet another embodiment, the biometric sensors 156 sense iris or facial identifying information. In some embodiments, the biometric sensors 156 incorporate lighting elements (not shown), such as LEDs (not shown) embedded beneath a clear outer detection cover (not shown) that can convey information through the application of a variety of colors, intermittent illumination regimens, or both, about the status of the Inhalation Device 110, including but not limited to its locked or unlocked status, approval of the provided identifying information via the biometric sensors 156, power supply 146 capacity and status, and estimated fluid amount remaining in installed and identified vaporization cartridge assemblies 118 and 118′.

When fully assembled, the Inhalation Device 110 allows for the pressurized communication of atmospheric air through the breather holes 152 and 152′ such that air and/or vapor exits the y-branched vapor venting conduit 148 at an outlet 158 (shown in FIGS. 7 & 9) within the mouthpiece 114.

FIG. 8 illustrates a bottom plan view of an embodiment of the present invention, which highlights one possible place and configuration of the breather holes 152 and 152′ in the bottom plate 150, along with the externally-accessible connection port 144. In another embodiment, the breather holes 152 and 152′ may instead comprise a single large vented opening with a mesh protector to filter any unwanted detritus from entering the Inhalation Device 110. The connection port 144 may be a USB port, MicroUSB port, MiniUSB port, firewire port, or any other standards-approved connection to the micro controller 138 that allows for the communication of data and the transmission of power to charge a rechargeable power supply 146 (shown in FIGS. 6, 7, & 9).

FIG. 9 illustrates a cross-sectional view of another twin-cartridge assembly embodiment of the present invention. Upon an application of negative pressure at the mouthpiece 114 (shown in FIG. 6) by a user, a negative pressure differential is created at the outlet 158 such that the fluidic communication of the atmospheric air throughout the Inhalation Device 10 starting at the breather holes 152 and 152′, allows air to flow separately from the surrounding atmosphere through the holes 152 and 152′, into each of the respective atomizer duty controllers 136 and 136′, up through the conductive tubular interfaces 134 and 134′, into the vaporizing elements 128 and 128′, and then through the internal vaporization transfer tubes 132 and 132′, through each contoured venting port 20 and 20′, to be finally recombined inside the y-branched vapor venting conduit 148, and finally down to the outlet 158 where it exits the Inhalation Device 110 via the mouthpiece 114. Valving (not shown) may ensure that fluid stored within the respective internal cavities 126 and 126′ of the fluid reservoir assemblies 122 and 122′ do not travel through the vaporizing elements 128 and 128′ up into the internal vaporization transfer tubed 132 and 132′ via the fluid interface ports 130 and 130′ upon the same application of negative pressure at the outlet 158.

Upon the same application of negative pressure at the outlet 158, the micro controller 138, having received confirmation of approved usage through the identifying information conveyed by the biometric sensors 156, and having confirmed that historical vaporization data recorded by the controller chip 140 and preset usage conditions set by either the user, a third party, or both, allows for the application of electrical current from the power source 146 to heat vaporizing heating elements (not shown) within each respective vaporizing elements 128 and 128′, to excite the fluid (not shown) within each vaporizing element 128 and 128′ in the presence of flowing air, thus creating vapor. As explained in the air-only context above, that same negative pressure would then pull the vapor out of each vaporizing element 128 and 128′, through the internal vaporization transfer tubes 132 and 132′, through the contoured venting ports 120 and 120′, into the y-branched vapor venting conduit 148, and finally down to the outlet 158 where it exits the Inhalation Device 110 via the mouthpiece 114 (shown in FIG. 6).

In one embodiment, the micro controller 138 may separately or jointly limit the duration of the application of electrical power from the power source 146 to each respective heating elements (not shown) in the vaporizing elements 128 and 128′. In an alternative embodiment, the micro controller 138 controls the transfer of electrical power from the power source 146 to the heating elements (not shown) in the vaporizing elements 128 and 128′, based on the preset parameters of the vaporization quality desired by the user, as indicated via a user interface accessible remotely in a software application, Internet browser, or other interface removed from the Inhalation Device 110 itself. In a further alternative embodiment, the micro controller 138 controls the transfer of electrical power from the power source 146 to the heating elements (not shown) in the vaporizing elements 128 and 128′, based on the preset parameters of the vaporization quality desired by the user. In a further alternative embodiment, the micro controller 138 receives instructions remotely from a user and/or third party via the wireless transceiver (not shown), which then allows for the measured timing control on the transfer of electrical power from the power source 146 to the heating elements (not shown) in the vaporizing elements 128 and 128′. In a further alternative embodiment, the micro controller 138 transmits sensor readings and calculated approximated status readings to a user and/or third party via the wireless transceiver (not shown), which then allows for the user and/or third party to appraise the vaporization use of the Inhalation Device 110 and determine whether dosing vaporization controls should be changed. In a further alternative embodiment, the micro controller 138 records sensor readings and compares to calculated approximated status readings to automatically regulate vaporization use of the Inhalation Device 110, per preset conditions and parameters defined by a user and/or third-party. In a further alternative embodiment, the micro controller 138 transmits sensor readings and calculated approximated status readings to a user and/or third party via the wireless transceiver (not shown), which then allows for the user and/or third party to appraise the vaporization use of the Inhalation Device 110 and determine whether dosing vaporization controls should be changed. In some embodiments, the micro controller 138 may also manage the actuation of sealing control valves (not shown) installed in the air-flow path within the Inhalation Device 110, such as within the interface points between the contoured venting ports 20 and 20′ and the y-branched vapor venting conduit 148, or the y-branched vapor venting conduit 148 and outlet 158 to regulate the flow of air or any other fluid through the device under predetermined criteria, regardless of the types of vaporization cartridge assemblies 118 or 118′ are installed. In another embodiment, the above-mentioned selection and regulation parameters controlled by the micro controller 138 may also apply to those embodiments of the present invention wherein more than two vaporizing cartridge assemblies 118 are installed within the Inhalation Device 110.

In an alternative embodiment, the vaporizing elements 128 and 128′ may include ultrasonic wave generators (not shown) in place of heating elements (not shown), such that fluid in proximate contact with the ultrasonic wave generator (not shown) converts to vapor upon application of electrical power from the power source 146 to the vaporizing elements 128 and 128′, as managed by the micro controller circuitry 138. This alternative embodiment has the advantage of limiting the risk of overheating or unwanted combustion resulting from generation of excess heat, as an ultrasonic wave generator generates only a minimal amount of heat in its operation. Additionally, depending on the nature of the installed vaporization cartridge assembly 118 at issue, each may employ a different vaporization methodology via their respective vaporization elements 128, such that that information is recognized and managed by the micro controller 138 via embedded identifiable markers (not shown) in each cartridge 118,

While the invention herein disclosed has been described by means of specific embodiments and applications thereof, numerous modifications and variations could be made thereto by those skilled in the art without departing from the scope of the invention set forth in the claims

What is claimed is:

1. A vaporization apparatus comprising:
a rigid body having at least one inlet, at least one outlet, and at least one inner cavity, wherein the inner cavity contains at least one cartridge having a reservoir containing a vaporizable-liquid;
at least one atomizer configured to vaporize the vaporizable-liquid;
a micro controller electrically coupled to the atomizer and a power source, wherein the micro controller includes at least one wireless transceiver; and
at least one biometric sensor coupled with the micro controller, wherein the micro controller controls the at least one atomizer to vaporize the vaporizable-liquid upon confirmation of an authorized user based on information from the biometric sensor, wherein the confirmation of the authorized user is communicated to the micro controller via the at least one wireless transceiver.

2. The vaporization apparatus of claim 1, wherein the at least one cartridge is removable.

3. The vaporization apparatus of claim 1, wherein the at least one cartridge is refillable.

4. The vaporization apparatus of claim 1, wherein the at least one cartridge includes an identifier recognizable by at least one corresponding identifying sensor coupled to the micro controller, and wherein the identifier is used by the micro controller o control activation of the at least one atomizer to vaporize the vaporizable-liquid.

5. The vaporization apparatus of claim 1, further comprising at least one vaporization control parameter communicated to the micro controller through the at least one wireless transceiver, wherein the at least one vaporization control parameter regulates activation of the at least one atomizer to vaporize the vaporizable-liquid.

6. The vaporization apparatus of claim 1, wherein the at least one vaporization control parameter a predetermined duration.

7. A measured dosing inhalation system comprising:
a hand-held vaporization device, comprising
at least one cartridge containing vaporizable fluid;
a micro controller having at least one wireless transceiver; and
at least one external biometric sensor coupled to the micro controller;
a computing device with computer program instructions configured to provide a graphic user interface to at least one individual, and communicatively coupled to the hand-held vaporization device via the at least one wireless transceiver, wherein the at least one individual may set at least one usage parameter of the hand-held vaporization device.

8. The measured dosing inhalation system of claim 7, wherein the at least one cartridge is removable from the hand-held vaporization device.

9. The measured dosing inhalation system of claim 7, wherein the at least one cartridge is refillable.

10. The measured dosing inhalation system of claim 7, wherein the at least one cartridge includes an identifier recognizable by at least one corresponding identifying sensor electrically coupled to the micro controller, and wherein the identifier is used by the micro controller to control activation of the vaporizable fluid.

11. The measured dosing inhalation system of claim 7, wherein the hand-held vaporization device further comprises at least one vaporization control parameter communicated to the micro controller through the at least one wireless transceiver, wherein the at least one vaporization control parameter regulates activation of the at least one atomizer to vaporize the vaporizable fluid.

12. The measured dosing inhalation system of claim 7, wherein the micro controller controls the hand-held vaporization device to vaporize the vaporizable fluid based on a predetermined duration value.

13. A method of configuring a vaporization device, comprising:
installing at least one cartridge having a reservoir containing vaporizable-liquid into a hand-held vaporization device by connecting the at least one cartridge to at least one electrical coupling configured to conduct electricity from a power source stored within the hand-held vaporization device to the at least one cartridge, wherein the at least one cartridge includes embedded identifying information recognizable by a micro controller within the hand-held vaporization device as being at least one defined characteristic of the at least one cartridge, and wherein the hand-held vaporization device includes at least one outlet and at least one inlet;
configuring the hand-held vaporization device to accept an identifier through at least one external sensor included on the hand-held vaporization device; and
configuring the hand-held vaporization device to operate in at least one preset duty cycle on the basis of the embedded identifying information of the at least one cartridge.

14. The method of claim 13, wherein the duty cycle comprises the duration of conduction of electricity from the power source to the at least one cartridge.

15. The method of claim 13, wherein the embedded identifying information comprises an RFID identifier installed within the at least one cartridge.

16. The method of claim 13, wherein the at least one defined characteristic comprises chemical characteristics and associated toxicity information.

17. A method of operating a vaporization device, comprising:
installing at least one cartridge having a reservoir containing vaporizable-liquid into a hand-held vaporization device, by connecting the at least one cartridge to at least one electrical coupling configured to conduct electricity from a power source stored within the hand-held vaporization device to the at least one cartridge, wherein the at least one cartridge includes embedded identifying information recognizable by a micro controller within the hand-held vaporization device as being at least one defined characteristic of the at least one cartridge;
configuring the hand-held vaporization device to accept an identifier through at least one external sensor included on the hand-held vaporization device;
configuring the hand-held vaporization device to operate in at least one preset duty cycle on the basis of the embedded identifying information of the at least one cartridge;
providing the identifier to the at least one external sensor; and
applying a lower pressure at the at least one outlet in comparison to present atmospheric conditions surrounding the hand-held vaporization device.

18. The method of claim 17, wherein the duty cycle comprises the duration of conduction of electricity from the power source to the at least one cartridge.

19. The method of claim 17, wherein the embedded identifying information comprises an RFID identifier installed within the at least one cartridge.

20. The method of claim 17, wherein the at least one defined characteristic comprises chemical characteristics and associated toxicity information.
